(19) United States
(12) Patent Application Publication
(10) Pub. No.: US 2023/0151321 A1
(43) Pub. Date: May 18, 2023

(54) FOOD WASTE DIGESTER

(71) Applicant: Minus Global Holdings Inc., Toronto (CA)

(72) Inventor: Louis Anagnostakos, Toronto (CA)

(73) Assignee: Minus Global Holdings Inc., Toronto (CA)

(21) Appl. No.: 17/455,273

(22) Filed: Nov. 17, 2021

(51) Int. Cl. C12M 1/00; B09B 3/00

(52) U.S. Cl. CPC C12M 45/00; B09B 3/00; C12M 43/00

(57) ABSTRACT

A method of processing food waste in a food waste digester using processed tagua seeds as a support medium for micro-organism that digest food waste into a biodegradable liquid.

### FIELD OF THE INVENTION

The present specification relates generally to food waste digestion, and more particularly to an improved food waste digester process.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

Food waste digesters are used in commercial operations where large quantities of food waste require disposal on a regular and continuous basis, such as restaurants or cruise ships. Generally, food waste digesters operate using aerobic micro-organisms to break down the food waste into a biodegradable and environmentally safe output product.

One requirement for waste digester is the need for a support medium for the micro-organisms in order to provide a stable living and growth environment within the digesters and inhibit the loss of micro-organisms during the waste digestion process. Generally, chips or beads of plastic are used to provide an inert medium. However, it has been found that, over time, the plastic may chip or shred, leading to plastic being released in the digester discharge. This plastic waste is undesirable for safety and environmental reasons. Accordingly, there is a need for improvement in the digester process to minimize or preferably eliminate this plastic waste.

Thus, it would be desirable to have a food waste digester and process which provides this improvement and/or mitigates some of the known disadvantages.

Accordingly, there remains a need for improvements in the art.

### SUMMARY OF THE INVENTION

In accordance with an aspect of the invention, there is provided a method of processing food waste in a food waste digester using processed tagua seeds as a support medium for micro-organism that digest food waste into a biodegradable liquid.

According to an embodiment of the invention, there is provided a method of processing food waste, comprising: inserting processed tagua seeds into a food waste digester, the amount of the tagua seeds determined according to an operating volume of the food waste digester; inserting food waste into the food waste digester; inserting bio-digesting micro-organisms into the food waste digester; and operating the food waste digester, wherein the bio-digesting micro-organisms reside, at least in part, upon the tagua seeds during operation. The resulting output of the food waste digester is a biodegradable liquid.

According to a further embodiment of the invention, the tagua seeds may be replenished on a scheduled basis, for example, the replenishment may take place monthly using an amount equal to one-third of the original amount of tagua seeds. Similarly, the micro-organisms may be replenished on a scheduled basis, such as bimonthly.

In some embodiments, the size of the tagua seeds is preferably between 0.75 and 1.5 inches in maximum diameter. The amount required is preferably one pound of tagua seeds per three gallons of volume in the food waste digester.

In some embodiments, the mixture of tagua seeds, micro-organisms and food waste is subject to agitation or mixing during operation of the food waste digester.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

Other aspects and features according to the present application will become apparent to those ordinarily skilled in the art upon review of the following description of embodiments of the invention in conjunction with the accompanying figures.

### BRIEF DESCRIPTION OF THE DRAWINGS

Reference will now be made to the accompanying drawings which show, by way of example only, embodiments of the invention, and how they may be carried into effect, and in which:

FIG. 1 is a block diagram of a food waste digester according to an embodiment; and

FIG. 2 is a schematic of a food waste digestion process according to an embodiment.

Like reference numerals indicated like or corresponding elements in the drawings.

### DETAILED DESCRIPTION OF THE EMBODIMENTS

The present invention relates to generally to food waste digestion, and more particularly to an improved food waste digester process.

“Food waste” in the present context is considered to be any plant or animal material, processed or unprocessed, which is generally fit for human consumption. Thus, meats, fruits, vegetables, eggs, pastas and breads are all food waste, as well as peels, stems, and trimmings from the same. However, bones and manufactured materials (paper, plastic, wood, metal) are not considered food waste and generally cannot be digested via the described process.

As discussed above, to date food waste digesters have used chips or beads of plastic as a support medium for micro-organisms with the food waste digester. These plastic chips have been found to shred or degrade over time, leading to the undesirable discharge of plastic as put of the food waste digester output. Accordingly, a replacement support medium is desirable.

Experimental data has determined the tagua seeds may be used as a replacement for the plastic chips used in known food waste digesters. The tagua seeds provide the advantage of being themselves biodegradable, however, they also require modification to the known operating procedures for the food waste digesters in order to provide equivalent or better performance.

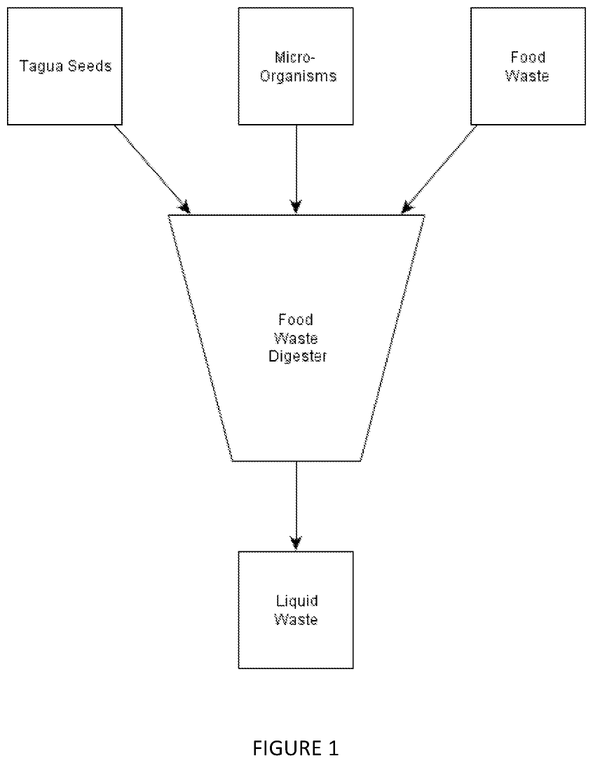

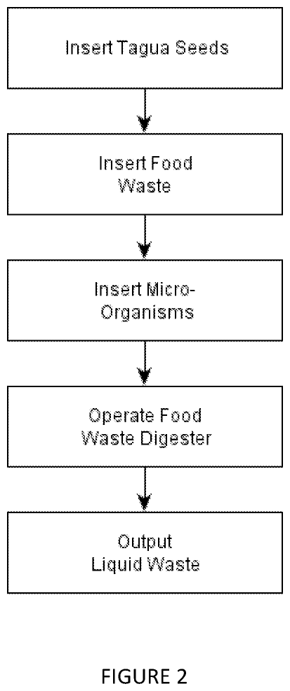

According to an embodiment as shown in FIGS. 1 and 2, initially, an amount of processed (de-hulled) tagua seeds in placed in the interior cavity of the food waste digester. The amount of tagua seeds initially used is dependent upon the interior cavity volume of the digester (see details below). Then, the food waste for processing is placed into the interior cavity, up to a maximum volume as indicated within the cavity. Finally, a volume of bio-digesting micro-organisms is added. The food waste digester is then ready for operation.

In operation, the micro-organisms aerobically digest the food waste in combination with oxygen from air introduced to the digester. Over time, the food waste is broken down until eventually turning into a biodegradable liquid, which may then be output and disposed of in the same manner as standard wastewater. A filtration screen (such as ˜0.5 mm mesh) may be used to prevent any significant solid food waste particles from exiting the digester.

A set of paddles operates continuously within the interior cavity to provide mixing and distribution of the micro-organisms throughout the food waste to ensure even digesting processes within the entire mixture.

Additional food waste may be added to the system over time as the volume in the digester is reduced, and new waste requires disposal. In most cases, the food waste may be added every 2-3 hours to maintain peak operating volume within the digester, and to address continuous waste production in food operations such as restaurants. Once operating, the food waste digester may remain continuously active subject to any maintenance or similar requirements.

As the digester operates over a period of time, the tagua seeds may gradually break down due to both physical agitation from the paddles, and from being digested by the micro-organisms as with the other organic matter in the digester. Accordingly, regular replenishment of the tagua seeds is required to ensure optimal digester operation. Testing data has demonstrated a deterioration rate of approximately 1% per day for the tagua seeds regardless of the digester volume, as the amount of tagua seeds and micro-organism scales with the digester volume. Accordingly, a replenishment schedule for the tagua seeds may be used to ensure a sufficient amount to maintain the digestion process at an optimal level remain present within the digester. Per testing, replenishment of the tagua seeds every 30 days by adding new tagua seeds in the amount of one-third of the original amount provided has shown to be sufficient to maintain operation.

Similarly, micro-organisms may require replenishing on an occasional basis, such as every 2 months, with an amount sufficient to ensure continued survival of the micro-organisms during operation of the digester.

In testing, after accounting for maintenance and other operational issues, food waste digester performance increased by as much as 200% over performance without any support medium. In operation, weekly waste amounts of 1200-2000 pounds were digested in four separate digesters without any support medium, and the weekly digested waste amounts increased to 2800-4000 pounds when the tagua seeds were added according to the methods disclosed herein. The testing units were approximately 450 gallons in overall interior volume and 150 pounds of tagua seeds were added as described above.

Other operational and maintenance steps may be required on either a routine (daily, weekly) or irregular (i.e. as-needed based on monitoring) basis. Such steps may vary based on the operating conditions and environment and may not be expected to modify any of the processes set out herein.

It should also be noted that the steps described in the method of use can be carried out in many different orders according to user preference. The use of “step of” should not be interpreted as “step for”, in the claims herein and is not intended to invoke any provisions of 35 U.S.C. § 112(f). It should also be noted that, under appropriate circumstances, considering such issues as design preference, user preferences, marketing preferences, cost, structural requirements, available materials, technological advances, etc., other methods are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the patent office and the public generally, and especially the scientist, engineers and practitioners in the art who are not familiar with patent or legal terms or phraseology, to determine quickly from a cursory inspection the nature and essence of the technical disclosure of the application.

The present invention may be embodied in other specific forms without departing from the spirit or essential characteristics thereof. Certain adaptations and modifications of the invention will be obvious to those skilled in the art. Therefore, the presently discussed embodiments are considered to be illustrative and not restrictive, the scope of the invention being indicated by the appended claims rather than the foregoing description and all changes which come within the meaning and range of equivalency of the claims are therefore intended to be embraced therein.

What is claimed is:

1. A method of processing food waste, comprising:
inserting processed tagua seeds into a food waste digester, the amount of the tagua seeds determined according to an operating volume of the food waste digester;
inserting food waste into the food waste digester;
inserting bio-digesting micro-organisms into the food waste digester; and
operating the food waste digester,
wherein the bio-digesting micro-organisms reside, at least in part, upon the tagua seeds during operation.

2. The method of claim 1, wherein the tagua seeds are sized between 0.75 to 1.5 inches diameter.

3. The method of claim 1, further comprising a step of replenishing the amount of tagua seeds on a scheduled basis.

4. The method of claim 3, wherein the tagua seeds are replenished on a monthly basis in an amount of one-third the original amount of tagua seeds.

5. The method of claim 1, wherein the amount of tagua seeds is one pound per 3 gallons of volume in the food waste digester.

6. The method of claim 1, further comprising a step of replenishing the amount of micro-organisms on a scheduled basis.

7. The method of claim 1, further comprising a step of outputting a biodegradable liquid formed from food waste digested by the bio-digesting micro-organisms.

8. The method of claim 1, further comprising agitating the mixture of tagua seeds, micro-organisms and food waste within the food waste digester during operation.
